(19) United States
(12) Patent Application Publication
(10) Pub. No.: US 2011/0002513 A1
(43) Pub. Date: Jan. 6, 2011

(54) Medical apparatus and method of using the same for generating a virtual road map guide of a patient's anatomy and annotate the images

(75) Inventors: Anthony Molinari, Grafton, MA (US); Mark Mariotti, Westford, MA (US); James Kiso, Dunstable, MA (US)

Correspondence Address: LAW OFFICES OF MICHAEL LECCESE, P.C., 790 TURNPIKE STREET, NORTH ANDOVER, MA, 01845, US

(73) Assignee: Foresight Imaging LLC, Chelmsford, MA (US)

(21) Appl. No.: 12/459,433

(22) Filed: Jul. 1, 2009

(51) Int. Cl. G06K 9/00; A61B 5/00

(52) U.S. Cl. 382/128; 600/407

(57) ABSTRACT

An apparatus for displaying and recording medical images and producing by sketching or drawing line segments creating an electronic virtual road map of a patient's anatomy for use by a physician during an intra-body medical procedure. The apparatus includes among its essential elements, an interventional device providing real time images of body structure of the patient; a medical image acquisition system computer adapted for acquisition of a plurality of medical imaging video signals wherein signals are acquired from a transmitting medical device in the medical device's native resolution having a central processing unit, a display, at least one input device, a storage device, an output device wherein the medical image is displayed on the display; and an image generating device, wherein physician uses the input device to sketch or draw line segments creating virtual road map of patient's anatomy over the medical image. The drawing features can also be used as a teaching tool, directing physicians as to what the plan for the procedure is. It can also be used to document the planned procedure.

A method of displaying and recording a medical image during an intra-body medical procedure, providing a medical image to a medical image acquisition system acquiring a plurality of medical imaging video signals wherein signals are acquired from a transmitting medical device in the medical device's native resolution having a central processing unit, a display, at least one input device, a storage device, an output device wherein the medical image is transmitting to a display, displaying the medical image on the display, viewing the medical image, the operator positioning the input device wherein a line segment appearing on the display, the operator, tracing segments using the input device, the segments appearing on the display over and along with the medical image creating curve segments coincident to the medical image.

### FIELD

The invention relates generally to a medical apparatus and method of using the same for generating a virtual road map guide of a patient's anatomy for use by a medical professional. The invention acquires medical images and through an input device the operator electronically sketches, annotates and stores a road map image overlaid on a captured medical image. Wherein creating a virtual road map, drawing or tracing of patient's anatomy and adding any additional information.

### BACKGROUND

Many medical procedures involve inserting an interventional device or typically a catheter into a patient for a variety of purposes. One such procedure is commonly known as cardiac catheterization used to obtain an angiogram. As part of this procedure a radiopaque dye or contrast agent is injected into the bloodstream and a radiogram is taken (better known as x-ray). An interventional device is any device that is introduced onto or into the human body. Normally the physician will insert the catheter into an artery in the human body and guide that catheter through the arterial system to the desired location. In order to position the catheter and monitor its location within the body of the patient, one or more x-ray type images may be taken prior to injecting dye for the angiogram. Angiography or arteriography is a medical imaging technique used to visualize the inside, or lumen, of blood vessels and organs of the body, with particular interest in the arteries, veins, vasculature and the heart chambers. The interventional device or catheter itself may also be visualized using the x-ray or fluoroscopy so the physicians can view the catheter and the patient's vasculature at the same time. As the physician moves the medical device (e.g. catheter) through the body of the patient he (or she) uses the x-ray or fluoroscopic image as presented on a display to help him guide the interventional device. The human vascular system contains many different paths, branches or courses to deliver blood to the body and the physician may only be interested in routing his interventional device in a certain way to get from the body insertion point to the point of interest. Therefore, it is critical for a successful procedure for the physician to see the vasculature, choose and document his path, or course, he wishes to take on the vasculature roadmap. Whether the physician uses a snapshot such as x-ray image to help him guide the catheter through the body or a fluoroscopic moving image to guide the catheter through the body the physician requires the image to help him guide the device. Briefly, fluoroscopy is commonly used and is an imaging technique to obtain moving real time images of a patient's internal structure. Both X-ray and fluoroscopy involves use of a form of ionizing radiation. Both carry potential health risks to the patient. The longer the patient is exposed to the radiation the more severe the potential health risk is to the patient, therefore it is advantageous to the patient to absorb as little radiation as possible. Often as the physician uses these interventional devices, the physician views images obtained via x-ray or fluoroscopy as it is being displayed on a display monitor during which or after the catheter procedure. The physician will use a static or real-time fluoroscopic image to aid in medical procedure, in most cases helping guide the interventional device to the desired location within the body. In many cases the physician would like the radiograph dye to be visualized on the fluoroscope for a period of time longer than it is.

During the medical device interventional procedure, the physician using his medical training (or medical device operator) will determine the best route to direct his interventional device through the patient's vasculature to treat or diagnose a patient. The physician with treatment in mind will determine the location of interest, such as, but not limited to routing a catheter to the location of the blood vessel blockage in order to treat the blockage. In most cases of a catheter based procedure, the physician would acquire an x-ray image or angiogram to determine patient anatomy and in many cases use a fluoroscopic image to feed the interventional device through the patient to the point of interest within the patient. Commonly, the patient's anatomy is visibly displayed through illumination techniques such as x-ray, fluoroscopic, and many different digital image enhancement techniques including, but not limited to digital subtraction angiography. Many of the imaging techniques use a radiopaque injected dye and radiation techniques so the physician can see the dye as it runs through the human body specifically as it is shown by x-ray and fluoroscopy medical techniques. The physician will use this image to determine the best location for routing of the interventional device to treat the patient. However, radiopaque dye only contrasts or enhances the blood vessels for a finite period of time. In most cases, the physician, as he (or she) routes the interventional device views images on a display monitor of the patient's anatomy and location of the device as it is passed through the patient. As the physician reviews the display or angiographic study, the physician may draw, sketch or trace directly on the display monitor to help him with the procedure or to help him describe or direct others in the procedure. In other words, while he views the complex vasculature of the patient with or without the catheter in place, he traces on the display screen over the medical image to help him guide and document the procedure. After the dye is gone the physician cannot see the vasculature and without more dye or a form of road mapping it is not possible to view the arterial vasculature system of the patient, so the physician makes these traces and annotations to help guide him. Presently the physician documents the location of critical aspects of this procedure by making some of these annotations directly on the display monitor, conceivably using a grease pencil or marking pen. Interventional and catheter based procedures performed with x-ray or fluoroscopy to generate angiographic images by means of ionizing radiation display the radiographic dye or enhanced vessel for only several seconds at a time. Therefore, creating a roadmap would enable the physician to route the device efficiently and safely and to not over expose the patient or physician operator to harmful radiation by use of the radioactive equipment for extended periods of time. Specifically, for example, the physician routing a catheter through the labyrinth of patients vascular structure will choose the best passage (to get from the percutaneous location on the body to the desired location within the vasculature structure) and because the structure is only visible on the monitor for a finite time, the physician will sketch or trace on the display monitor as the procedure is performed. This way the physician can trace his route, notes and document his path to view his route or road map even after the dye contrast wears off.

Many physicians will trace a vascular map on the display screen or a transparent film taped on or mounted over the display screen allowing the physician to view the vascular placement of the catheter in real-time as the procedure progresses. The physician will trace, mark, draw a road map, sketch a placement and perhaps annotate on the transparent film or screen or monitor as it resides over the display. However because the opacified blood image is only viewable for seconds the physician may take and view multiple images before he traces on the overlay or monitor, he perhaps will choose an image (at that point in time a non enhanced fluoro image, non subtracted fluoro image) and trace on the overlay or monitor. The overlay tracing may be used to track the catheter location and movements relative to the vascular map. Also, the physician traces the map on the display in the event the patient moves. In other words, if the catheter location is being tracked on the screen without a trace and the patient moves, the physician may lose track of his reference point and may find it necessary to repeat the process resulting in unwanted higher doses of radiation. Tracing or drawing overlay method presently used by many physicians comes with many disadvantages, some of which include no digital permanent record of physicians tracings and instruction, different displays have different image quality and different displays have different parallax effect resulting in misinformation as originally intended.

It would be desirable to have a simple cost-effective efficient apparatus and process for the physician to trace, record and add annotations digitally viewing and documenting the location of the catheter or other device over the viewed X-ray and/or fluoroscopic image. The inventor has identified the need and usefulness for such an invention described herein.

The traditional way of capturing an image on a medical imaging machine commonly called a modality, generally consisted of an operator or technician first conducting a scan. Then using the modality to save the image, in still or motion video format, into the modality memory or into a main image storage database. Soon afterward perhaps downloading the image into a hospital database such as a PACS system, Picture Archiving and Communications System (or PACS), for storage and later retrieval. The doctor would then access the PACS system to retrieve the image, the doctor at that time would call up the image, view and review the image and conceivably develop a diagnosis based on the information from the image. This system imagery can accomplish all these tasks in real time. The illustrated embodiment is comprised of two software application programs, one called Tele Medicine Imagine Management System (TIMS), the other called TIMS Fluoro-TRACE™. TIMS is a server type program on a single computer that is acquiring the medical video signal and transmitting video and voice signals to a client program. The TIMS Fluoro-TRACE™ system (i.e. this invention described herein) allows for the physician to input highlighted graphic electronic traces over the original medical image.

In one embodiment, TIMS provides the real-time video and audio communication as well as a method of recording and transmitting images in DICOM format. DICOM, Digital Imaging and Communications in Medicine, is a medical imaging standard common in the medical industry. The embodiment can serve as the connection point between any medical imaging system and a hospital PACS, patient archival and communications system. One capability of the TIMS program is to connect older non-DICOM equipment to a hospital network, allowing imaging studies to be stored in PACS. It can also be used to connect DICOM compatible equipment, if it desired, to use some features contained in TIMS that are not available on the imaging system such as the TIMS Consultant feature, and the TIMS Fluoro-TRACE™. The TIMS Fluoro-TRACE™, or this invention described herein as a trace overlay can be stored with the DICOM data and viewable in PACS.

In yet another embodiment illustration of this invention, the physician imputed drawing or tracing can be contained within a direct digital medical acquisition instrument. In many cases medical-imaging instruments will contain their own computer system. The direct digital acquisition system can acquire and store its own digital images. In this system there is no Tele-Medicine Management System (no external computer, no external added data management software, no analog to digital converter) or no need to convert any analog signal to a digital one. The direct digital medical acquisition system may contain all the necessary equipment to operate as a computer so will include its own CPU, storage device, RAM, connections, operating system software, application software and the like. Therefore, the physician merely uses the direct digital medical acquisition instrument having this invention loaded therein, software application to display and record medical images, an input device allowing the operator to create a line segment drawing over the displayed image and saving the image with line segment drawing. In addition to the location of the catheter for an angiogram, there are many other medical procedures where an interventional device is inserted into a patient, such as but not limited to endoscopy.

In most, if not all such procedures, locating, positioning and recording the interventional device with a patient is extremely important.

In the past many other inventors have seen the need to solve the problem of tracking and documenting the position of the invention devices as it is in the body and have solved this problem in other ways. Prior art U.S. Pat. No. 6,515,657 discloses a volumetric ultrasonic imaging system that superimposes section views created from volumetric ultrasonic image data and location data for intervention device, such as a catheter. The position of the intervention device may be shown, in one or more views relative to organ and tissues with a body as the intervention device is moved. The intervention device is positional dated is updated continuously and is superimposed on tissue image data that may be updated less frequently. Generally most prior art addressing the problem of locating and tracking an intervention device within the human body has been found to be inefficient, costly, cumbersome and not necessarily desired by most physicians. Many different prior arts disclose many different apparatuses and methods of tracking such devices and many different ways to enhance medical images to aid the physicians in their tasks. To date apparatus and methods of tracking and documenting such a procedure focus on having transmitting and receiving equipment to locate catheter tracking such as prior art U.S. Pat. No. 6,403,577 and U.S. Pat. No. 6,226,543. Others use image techniques to enhance digital image quality such as U.S. Pat. No. 7,233,689.

Prior art also discloses many different inventions for apparatus and method of electronic sketching, electronic sketchpad and electronic free hand sketching. These arts teach many different ways to use electronic means or computers to sketch, draw or draft line segments on a computer display using an input device. Typically the input device can be a stylus, a mouse, touch pad or touch screen. Other input devices include but are not limited to keyboards, touch screens, touch pad, trackballs, light pens, graphics tablet and joysticks. All input devices mentioned herein are operated by the human hand or human finger, such as movement of a computer mouse or by touching a screen or touch pad. It is common in the art sketching segments or line segments sometimes know as computer aided drafting or computer aided design. Among the many prior arts include, U.S. Pat. No. 4,764,763 An Electronic Sketching Device, and U.S. Pat. No. 6,233,351 Method and Apparatus for Processing a Freehand Sketch. However no prior art technology allows a physician to trace electronically line segments and notations over newly acquired or existing medical imagery.

The inventor has developed a novel and simple apparatus and method to allow a physician to trace, annotate and store images while using medical intervention technologies.

### SUMMARY

The invention relates generally to a medical apparatus and method of using the same for generating a virtual road map guide of a patient's anatomy for use by a medical professional while conducting a procedure on the patient. The apparatus including a medical image acquisition system adapted for receiving and transmitting medical images, constructed from, a computer having communications capability adapted for acquisition and transmission of a plurality of medical imaging video signals wherein the video signals are acquired from a transmitting medical device in the medical device's native resolutions, transmitting the signals at their native resolutions to a receiving device receiving the medical imaging video signals in analog or digital source and if required compressing and/or scaling the signal, converting the signal to digital form for transmission, and transmitting the digital signals to a display device. The medical image acquisition system is capable of acquiring signals from a plurality of medical imaging systems including but not limited to, ultrasound, Computer Tomography (CT) scan, fluoroscopy, endoscopy, magnetic resonance imaging, nuclear medicine, echocardiogram ultrasound and microscopy. The medical receiving device receiving the video image signal in a plurality of video sources including but not limited to, S-video, composite color and monochrome, component red blue green video (RGB, three additive primary colors), digital visual interface (DVI, digital standard interface) and high definition multimedia interface (HDMI, compact audio video interface uncompressed digital data), serial digital interface (SDI, serial digital interface) and DICOM video. The apparatus including a storage device adapted for archiving the video signal in a predetermined digital format including Digital Imaging and Communications for Medicine (or DICOM), Audio/Video Interleaved. Transmitting data includes transmitting the data in secure encryption protocols. Transmitting video signal resolution at the same resolution as the received signal. In one illustration, a remote location communicates with the networked computer, for the purpose of collaborating and conferencing.

An apparatus and method for using a medical image acquisition system for receiving, transmitting and road mapping medical image data, following the steps of acquiring image data from an image producing medical device, receiving medical video signals into a computer converter, compressing the video signals, converting the video signals to digital images, the medical image acquisition system connecting transmitting streaming image data instantaneously, the operator using an input device sketching lines, draws arcs, line segments, shapes and annotations over the medical video signals the medical acquisition system storing the image data.

An example or illustration of use may be, as the physician is viewing the image on the display, specially viewing a fluoroscopic image of the patient's vascular image possibly having a catheter in the patient, the physician can use the input device to trace or draw line segments to highlight the vascular path he wishes to take or merely to document or record the present location of the catheter within the patient. In addition the physician may also use the input device to create lines, line segment or annotations, to callout, highlight or map specific locations or points of interest such as but not limited to where he wishes to place a stent within the patient's vasculature. These drawing features can also be used as a teaching tool, directing physicians as to what the plan for the procedure is. It can also be used to document the planned procedure. The physician can also use these drawing features to draw a land mark such as on the ribs or vertebral body in the event the intervention device (image intensifier) must be moved during the procedure then repositioned later to be in line with the original roadmap.

The hardware input device sends information into the computers central processing unit (CPU). For example when an operator moves the mouse on a flat surface in an X-Y motion (left, right, up, down) a line segment will appear on the computer display (light pixels on the display illuminate to depict a viewable segment on the display). The line segments will appear in a changeable contrasting color to the original viewed medical image. The physician can also annotate or sign his name using the input device. Basic touch screen mechanics can also mimic computer mouse depress (click) and drag features. Characteristically the operator of the computer input sometimes referred to as a mouse can depress or click and hold the button while moving the mouse can create the line segment. Capturing the freehand line segment positions and storing the overlay typically as a one bit bitmap computer file format. The DICOM standard provides tags for storing this data The highlighted image of line segments may look like a tree and branch like structure as the human vascular structure may look like or sometime know as highlighting a road map through the vascular. The physician or operator can also erase or clear any, line segment, line, curve, drawing, trace, entire screen or annotation. Typically the physician can draw by depressing or clicking the input device such as a computer mouse button that toggles drawing or erasing. The operator can also use the input device to clear the entire entry.

The principle preferred embodiment and modes of operation of the present invention have been described in the forgoing specification. The invention which is intended to be protected herein, however, is not to be construed as limited to the particular embodiments disclosed, since these embodiments are to be regarded as illustrative rather than restrictive. Variations and changes may be made by others without departing from the spirit of this invention. Accordingly, it is expressly intended that all such variation and changes which fall within the spirit and scope of the claims be embraced thereby.

### BRIEF DESCRIPTION OF DRAWINGS

Other objects, features, and advantages will occur to those skilled in the art from the following description of an embodiment and the accompanying drawings, in which:

FIG. 1, shows a screen shot of an image of human vasculature with catheter

FIG. 2, shows a screen shot of an image of human vasculature with catheter having highlight imagery

FIG. 3, shows block diagram of typical input, computer, and output

FIG. 4, shows a screen shot of embedded highlighted image with other images

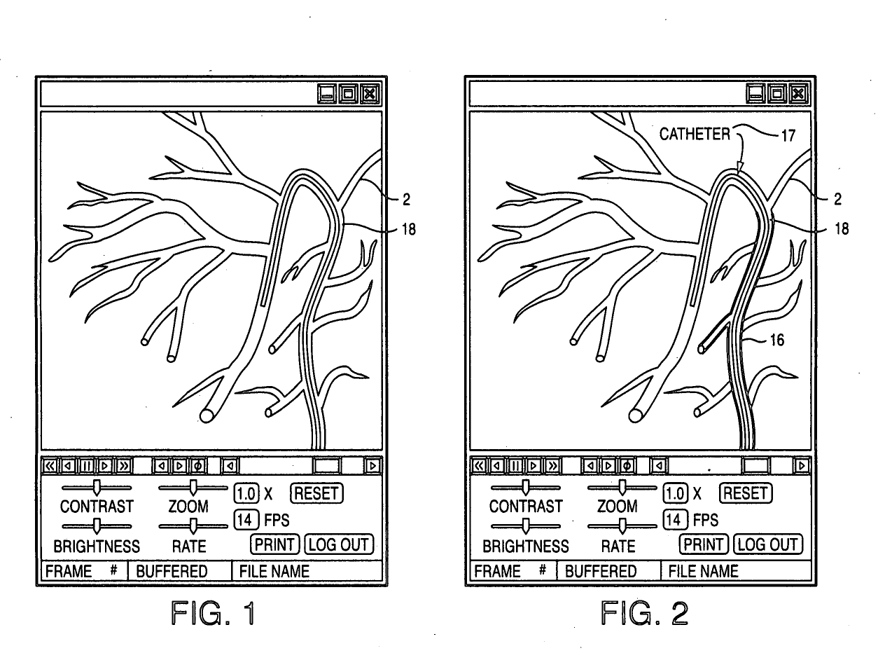

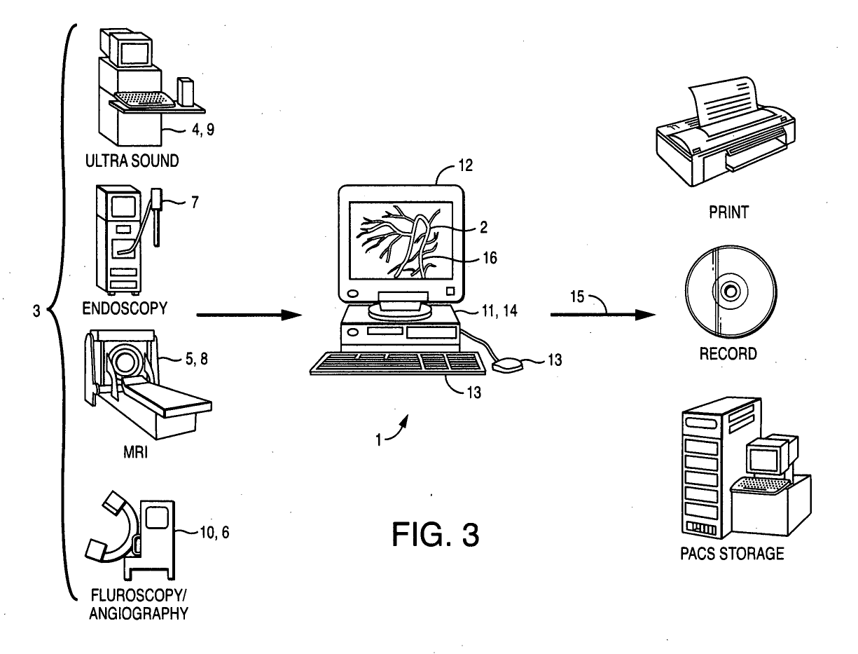

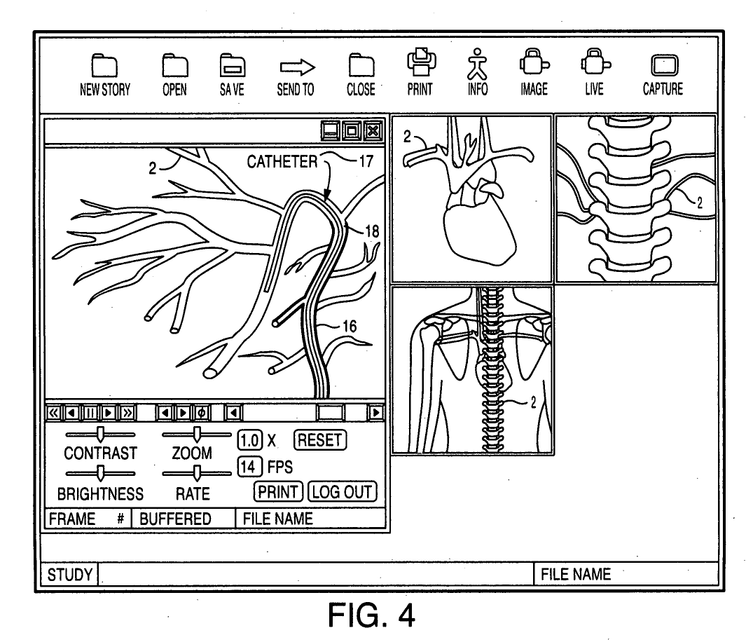

### DETAILED DESCRIPTION

An apparatus medical image acquisition system for displaying and recording medical images 1 and producing a virtual road map 16 of a patients anatomy 2 for use by a physician during an intra-body medical procedure. The medical image acquisition system having an intervention device 3 acquiring system signals from a plurality of medical imaging systems including but not limited to; ultrasound 4, Computer Tomography (CT) scan 5, fluoroscopy 6, endoscopy 7, magnetic resonance imaging 8, nuclear medicine, echocardiogram ultrasound 9, angiograph 10 and microscopy. The medical image acquisition system computer 1 adapted for acquisition of a plurality of medical imaging video signals wherein signals are acquired from a transmitting medical device, 4,5,6,7,8,9,10 in the medical device's native resolution having a central processing unit 11, a display 12, at least one input device 13 such as a computer mouse, stylus pen, track ball, tablet or touch screen display, a storage device 14 that is typically a computer having a central processing unit and file storage media such as a hard drive, an output device such as a display monitor or printer 15 wherein the medical image is displayed on the display 12 (or monitor), bus bar connections, operating software and application software. The image generating device 1 for generating for a virtual road map 16, wherein the virtual road map 16 comprises a two dimensional image illustrations representing anatomy structure as imputed by and operator 16. The apparatus for displaying and recording medical images 1 and producing a virtual road map 16 of a patient's anatomy for use by a physician during an intra-body medical procedure where the illustration represents operators input annotations 17.

The apparatus for displaying and recording medical images 1 and producing a virtual road map of a patient's anatomy 16 for use by a physician during an intra-body medical procedure where in the illustration represents vascular structure 2. In addition the medical image also can show the location of the intervention device such as a catheter 18.

A method of displaying and recording a medical image 1 during an intra-body medical procedure providing a medical image to a medical image acquisition acquiring a plurality of medical imaging video signals wherein signals are acquired from a transmitting medical device 4,5,6,7,8,9,10 in the medical device's native resolution having a central processing unit 11, 14, a display 12, at least one input device 13, a storage device 14, an output device wherein the medical image is displayed on the display 12; displaying the medical image on the display, viewing the medical image, the operator positioning the input device 13 wherein a line segment 16 appearing on the display 12, the operator, tracing segments 16 using the input device 13, the segments appearing on the display 12 resembling a road map 16, creating curve segments coincident to the medical image 2.

What is claimed is:

1. An apparatus for displaying and recording medical images and producing a virtual road map of a patient's anatomy for use by a physician during an intra-body medical procedure, comprising;
an interventional device providing real time images of body structure of the patient;
a medical image acquisition system computer adapted for acquisition of a plurality of medical imaging video signals wherein signals are acquired from a transmitting medical device in the medical device's native resolution having a central processing unit, a display, at least one input device, a storage device, an output device wherein the medical image is displayed on the display; and
an image generating device for generating for a virtual road map, wherein the virtual road map comprises a two dimensional image illustrations representing anatomy structure as imputed by an operator.

2. An apparatus for displaying and recording medical images and producing a virtual road map of a patient's anatomy for use by a physician during an intra-body medical procedure as in claim 1 wherein the illustration represents operator's input annotations.

3. An apparatus for displaying and recording medical images and producing a virtual road map of a patient's anatomy for use by a physician during an intra-body medical procedure as in claim 1 where in the illustration represents vascular structure.

4. An apparatus for displaying and recording medical images and producing a virtual road map of a patient's anatomy for use by a physician during an intra-body medical procedure as in claim 1 wherein the medical images are pre-recorded images.

5. An apparatus for displaying and recording medical images and producing a virtual road map of a patient's anatomy for use by a physician during an intra-body medical procedure as in claim 1 wherein the virtual road map is sketched line segments.

6. An apparatus for displaying and recording medical images and producing a virtual road map of a patient's anatomy for use by a physician during an intra-body medical procedure as in claim 5 wherein the virtual road map is sketched line segments chosen from a viable pallet of colors and line weights.

7. A method of displaying and recording a medical image during an intra-body medical procedure, comprising;
providing a medical image to a medical image acquisition system acquiring a plurality of medical imaging video signals wherein signals are acquired from a transmitting medical device in the medical device's native resolution having a central processing unit, a display, at least one input device, a storage device, an output device wherein the medical image is transmitting to a display;
displaying the medical image on the display, viewing the medical image, the operator positioning the input device wherein a line segment appearing on the display, the operator, tracing segments using the input device, the segments appearing on the display over and along with the medical image creating curve segments coincident to the medical image.
